(19) United States
(12) Patent Application Publication
(10) Pub. No.: US 2026/0262934 A1
(43) Pub. Date: Sep. 10, 2026

(54) HIGH-RESOLUTION SLIT LAMP ANGULAR MEASUREMENT ADD-ON DEVICE

(71) Applicant: Parsa BarazandehNoveyri, Ottawa (CA)

(72) Inventor: Behzad BarazandehNoveyri, Mashhad (IR)

(21) Appl. No.: 19/556,451

(22) Filed: Mar. 4, 2026

Related U.S. Application Data
(63) Continuation-in-part of application No. 18/459,324, filed on Aug. 31, 2023, now abandoned.
(60) Provisional application No. 63/402,527, filed on Aug. 31, 2022.

(51) Int. Cl. A61B 3/00; A61B 3/135; G01D 5/12

(52) U.S. Cl. CPC A61B 3/0025; A61B 3/135; G01D 5/12

(57) ABSTRACT

The present disclosure provides a high-resolution angle measuring device couplable to ophthalmic systems to determine the precise angle of rotation when using the ophthalmic system. The high-resolution angle measuring device enables accurate measurement of the horizontal angle of rotation of a slit beam from a slit lamp system (precise angles over the cornea of a patient). The proposed angle measuring device provides a compact, affordable and easy-to-use device able to measure and determine angular change of devices it is coupled to and used with.

### PRIORITY AND REFERENCE

This application is a continuation-in-part of U.S. patent application Ser. No. 18/459,324 filed Aug. 31, 2023, now pending, that claims priority to U.S. provisional patent application 63/402,527 filed Aug. 31, 2022, the contents of which are hereby incorporated by reference.

### TECHNICAL FIELD

This invention relates in general to ophthalmic instruments and more specifically to slit lamps.

### BACKGROUND

This section is intended to provide a background or context to the invention that is recited in the claims. The description herein may include concepts that could be pursued but are not necessarily ones that have been previously conceived or pursued. Therefore, unless otherwise indicated herein, what is described in this section is not prior art to the description and claims in this application and is not admitted to be prior art by inclusion in this section.

The slit lamp is an instrument consisting of a high-intensity light source that can be focused to shine a thin sheet of light into the eye. The lamp facilitates an examination of the anterior segment and posterior segment of the human eye, which includes the eyelid, sclera, conjunctiva, iris, natural crystalline lens, and cornea. A binocular slit-lamp examination provides a stereoscopic magnified view of the eye structures in detail, enabling anatomical diagnoses to be made for a variety of eye conditions.

A second, hand-held lens can be used to examine the retina. However, slit lamps typically do not provide accurate and reliable angular measurement of the patient eye and have to be used conjointly with other electronic devices which makes it both expensive and somehow impractical as the physician would not be able to measure and mark the angle accordingly.

Briefly described, the angular measurement of or on the eye may be needed in preparation for an ophthalmic procedure, during the procedure to confirm the accuracy of the steps being taken by the surgeon, or as a part of the post-operation services a patient receives. For example, in Toric IOL implant (which refer to astigmatism correcting intraocular lenses used at the time of cataract surgery to decrease post-operative astigmatism), the angular location of the phaco incision has to be measured to prepare the patient for many ophthalmic procedures.

However, the regular slit lamps do not provide an accurate and reliable angular measurement and the devices used for this purpose are typically expensive and complicated.

Furthermore, the accuracy of such measurement is quite important. For example, it has been estimated that “for every degree of misalignment, about 3 percent of the lens cylinder power is lost,” [Toric IOLs: Nailing The Alignment, Retrieved:

https://www.reviewofophthalmology.com/article/toric-iols-nailing-the-alignment], therefore a 30-degree misalignment would actually result in an increase of the patient's postoperative astigmatism.

As mentioned before, the inaccurate measurement of the angle of Toric lenses inside the eye reduces the effect of Toric intraocular lenses and ultimately leads to a decrease in the patient's vision. This may result in the need to correct the position of the lens after the operation, so that the patient's vision reaches its maximum, and otherwise, it causes the need for glasses.

On the other hand, the existing measurement methods are time consuming, which excludes the physician's possibility of performing a higher amount of surgery due to the interruption in the operation time.

There exists a need for a device and methods capable of accurately determining the axis of cylindrical refractive power in the cornea to which the Toric IOL implant should be aligned. In addition, there is a need for systems and methods that allow for more accurate positioning of the Toric IOL implant at the optimal angular orientation for correction of the astigmatic power of the cornea.

Additionally, slit lamps are equipped with low-resolution analog protractor that the operator must use to determine (i.e., approximate) the horizontal angle of the slit, which results in inaccurate and unreliable measurements/values of horizontal angle of the slit.

Therefore, there exists a need for a device to provide high accuracy/precision angular measurements of the horizontal angle of rotation of the slit when testing the patient with a slit lamp.

### SUMMARY

The present disclosure provides a high-resolution angle measuring device couplable to ophthalmic systems to determine the precise angle of rotation when using the ophthalmic system. The high-resolution angle measuring device enables accurate measurement of the horizontal angle of rotation of a slit from a slit lamp system (precise angles over the cornea of a patient). The accurate angle measured thereby may be used to determine a desired ocular implant or incisions, which may be important for Toric IOL implants.

The proposed angle measuring device provides a compact, affordable and easy-to-use device able to measure and determine angular change of devices it is coupled to and used with.

One broad aspect of the present disclosure is an angle measuring device couplable to and for use with a slit lamp of the type having a slit light source producing a slit beam, the slit light source being mounted to be rotatable about a vertical axis, the slit lamp having a mirror to direct the slit beam horizontally with rotation of the slit beam being about a horizontal axis, the angle measuring device for use in determining an angle of rotation of the angle measuring device indicative of a horizontal angle of rotation of the slit beam, the angle measuring device comprising: (1) a housing comprising: (1a) sensors comprising a Micro-Electro-Mechanical Systems (MEMS) gyroscope paired with MEMS accelerometers for measuring data comprising information about angular velocity and acceleration of the angle measuring device; and (1b) a microcontroller connected the sensors for determining the angle of rotation of the angle measuring device based on the data measured by the sensors; (2) a coupling member shaped to be coupled to a light source outer body of the slit light source of the slit lamp and for supporting the housing; and one of the group consisting of: (3a) a display on the housing for displaying the determined angle of rotation; (3b) a wireless data transceiver for providing communication to a user's device for at least transmitting data about the determined angle of rotation; and (3c) a wired connection interface for outputting data about the determined angle of rotation.

In some embodiments of the angle measuring device, the coupling member is shaped to fit at least partially over or around the light source outer body.

In some embodiments of the angle measuring device, the coupling member comprises a ring-shaped portion for removably coupling to the light source outer body and housing coupling portion for coupling to and supporting the housing.

In some embodiments of the angle measuring device, the angle measuring device comprises the data transceiver, wherein the data transceiver uses Bluetooth® technology.

In some embodiments of the angle measuring device, the angle measuring device comprises the data transceiver, and wherein the angle measuring device further comprises a pairing button for indicating to the microcontroller that the user's device is to be wirelessly connected.

Some embodiments of the angle measuring device further comprise a zeroing button for indicating to the microcontroller a current angular value corresponds to a reference value.

In some embodiments of the angle measuring device, a Kalman filter is associated with the microcontroller for determining the angle of rotation of the angle measuring device.

In some embodiments of the angle measuring device, the sensors comprise a nine-axis accelerometer.

In some embodiments of the angle measuring device, the coupling member is removably couplable to the outer body or removably couplable to the light source outer body of the slit light source of the slit lamp.

In some embodiments of the angle measuring device, the coupling member comprises a base mounting having a shape to receive, accommodate or match the shape of the light source outer body.

In some embodiments of the angle measuring device, the sensors comprise an inertial measurement unit (IMU).

Some embodiments of the angle measuring device further comprise a signal filter connected to the sensors to receive the measured data and connected to the microcontroller to provide at least a yaw angular rotation of the angle measuring device.

Another broad aspect of the present disclosure is a system for determining a horizontal angle of rotation of a slit beam of a slit lamp, the system comprising: (1) a slit lamp of the type having a slit light source producing the slit beam, the slit light source being mounted to be rotatable about a vertical axis, the slit lamp having a mirror to direct the slit beam horizontally with rotation of the slit beam being about a horizontal axis; (2) an angle measuring device couplable to the slit lamp, the angle measuring device comprising: (2a) a housing comprising: (2a.i) sensors comprising a Micro-Electro-Mechanical Systems (MEMS) gyroscope paired with MEMS accelerometers for measuring data comprising information about angular velocity and acceleration of the angle measuring device; and (2a.ii) a microcontroller connected the sensors for determining the angle of rotation of the angle measuring device based on the data measured by the sensors; (3) a coupling member shaped to be coupled to a light source outer body of the slit light source of the slit lamp and for supporting the housing; and one of the group consisting of: (4a) a display for displaying the determined angle of rotation of the angle measuring device, (4b) a wireless data transceiver for providing communication to a user's device for at least transmitting the determined angle of rotation of the angle measuring device, and (4c) a wired connection interface for outputting the determined angle of rotation of the angle measuring device; and (5) a user's device for receiving, from the angle measuring device, data about the angle of rotation determined by the microcontroller of the angle measuring device and comprising a processor and memory comprising executable program code that, when executed by the processor, causes the processor to provide a user interface displaying the angle of rotation determined by the microcontroller of the angle measuring device.

In some embodiments of the system, the program code, when executed by the processor, further causes the user's device to activate an angle zeroing function of the angle measuring device so a current angular value determined by the angle measuring device corresponds to a reference value.

Yet another broad aspect of the present disclosure is a method of determining an angle of rotation of a slit light source of a slit lamp indicative of a horizontal angle of rotation a slit beam of the slit lamp, the method comprising: (1) providing the slit lamp, wherein the slit lamp is of the type having the slit light source producing the slit beam, the slit light source being mounted to be rotatable about a vertical axis, the slit lamp having a mirror to direct the slit beam horizontally with rotation of the slit beam being about a horizontal axis; (2) providing a Micro-Electro-Mechanical Systems (MEMS) gyroscope and MEMS accelerometers; (3) coupling the MEMS gyroscope and MEMS accelerometers to the slit light source; (4) determining an initial orientation of slit light source corresponding to a reference orientation of the slit lamp; (5) rotating the slit light source around the vertical axis to change an orientation of the slit beam; (6) using the coupled MEMS gyroscope and MEMS accelerometers to measure the angular velocity and acceleration during the rotating of the slit light source around the vertical axis; and (7) using the measured angular velocity and the measured acceleration to determine the change of orientation of the slit light source of the slit lamp indicative of the horizontal angle of rotation the slit beam of the slit lamp.

In some embodiments of the method, the step of determining the initial orientation of slit light source comprises positioning the slit light source to the initial orientation and zeroing the MEMS gyroscope and the MEMS accelerometers.

Some embodiments of the method further comprise recording the determined change of orientation.

In some embodiments of the method, the recording the determined change of orientation comprises saving in memory data comprising information indicative of the determined change of orientation.

Some embodiments of the method further comprise associating the recorded change of orientation with a patient.

Some embodiments of the method further comprise associating the data with a digital medical file of the patient.

### BRIEF DESCRIPTION OF THE DRAWINGS

The invention will be better understood by way of the following detailed description of the embodiments of the invention with reference to the appended drawings, in which:

FIG. 1 shows a schematic perspective view of a state-of-the-art slit lamp;

FIG. 2A presents a perspective view of one possible embodiment of the high-resolution angle measuring device couplable to and for use with slit lamps;

FIG. 2B presents a front view of the high-resolution angle measuring device of FIG. 2A;

FIG. 2C presents a side view of the high-resolution angle measuring device of FIG. 2A;

FIG. 3A presents a side view of the high-resolution angle measuring device of FIG. 2A when installed on the slit lamp of FIG. 1;

FIG. 3B presents a perspective view of another possible embodiment of the high-resolution angle measuring device when affixed (e.g., glued) to the slit lamp of FIG. 1;

FIG. 4 is a block diagram of the various components of one possible embodiment of the high-resolution angle measuring device couplable to and for use with slit lamps; and

FIG. 5 presents one possible embodiment of one of the user interfaces that may be provided by a mobile application for use by a user's device to record the readings of the high-resolution angle measuring device.

FIG. 6 shows a flowchart of a method of determining the angle of rotation of the slit beam of the slit lamp of FIG. 1, which the angle measuring device can enable.

FIG. 7 presents a block diagram of one embodiment of a system comprising the slit lamp, the angle measuring device and a user's device.

### DETAILED DESCRIPTION

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

From the foregoing it will be appreciated that, although specific embodiments have been described herein for purposes of illustration, various modifications may be made without deviating from the spirit and scope of the teachings. Accordingly, the claims are not limited by the disclosed embodiments.

The following is a detailed description of embodiments of the disclosure depicted in the accompanying drawings. The embodiments are in such detail as to clearly communicate the disclosure without limiting the anticipated variations of the possible embodiments and may encompass all modifications, equivalents, combinations and alternatives falling within the spirit and scope of the present disclosure. It will be appreciated by those skilled in the art that well-known methods, procedures, physical processes and components may not have been described in detail in the following so as not to obscure the specific details of the disclosed invention.

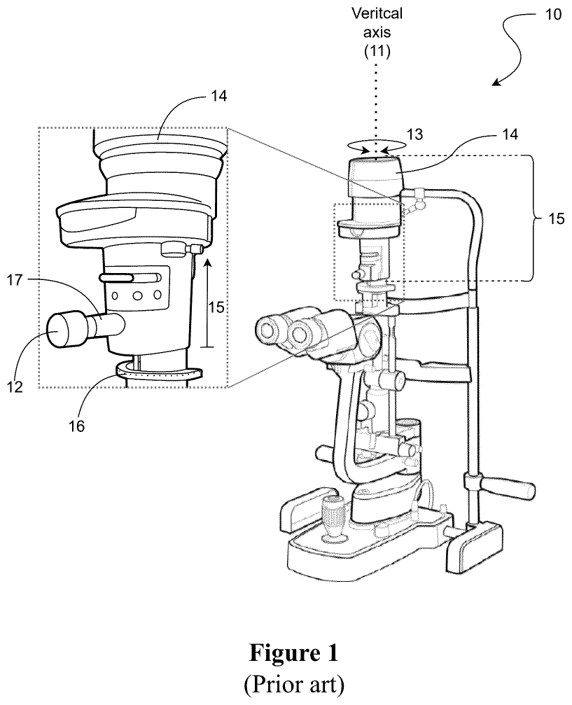

FIG. 1 shows a state-of-the-art slit lamp 10 (e.g., similar to the Slit Lamp SL-D7 or SL-D8Z. The slit lamp 10 may be of the type having a slit light source 15 producing a slit beam. The slit light source 15 being mounted to be rotatable a desired orientation/angle 13 about a vertical axis 11, the slit lamp having a mirror to direct the slit beam horizontally with rotation of the slit beam being about a horizontal axis (i.e., the desired orientation 13 corresponds to the horizontal angle of rotation of a slit beam).

The slit lamp 10 may comprise a light source housing 14 for hosting a high-intensity light source that can be focused into a slit beam (i.e., a thin focuses sheet of light) to be projected into the eye of the patient. The slit lamp 10 includes a top section 15 (corresponding to the slit light source) that can be rotated around the remainder of the lamp at a desired orientation/angle 13, which may correspond to the horizontal angle of rotation of a slit beam selected by the operator of the slit lamp 10. The slit lamp 10 comprises a control lever 17 for rotating the top section 15 of to a selected horizontal angle 13 of the slit beam. An aperture/slit-length control knob 12 may be located on the control lever 17. In some embodiments, the top section 15 of the slit lamp 10 is located above a low-resolution analog protractor 16 fix on the remainder of the slit lamp 10 to determine the angle between the top section 15 and the remainder.

Due to the low-resolution (e.g., 5-degree resolution) of the analog protractor, the angular measurements of the horizontal angle 13 of the slit beam that operators make are highly likely to be inaccurate and unreliable, which may result in medical errors or mistakes down the line.

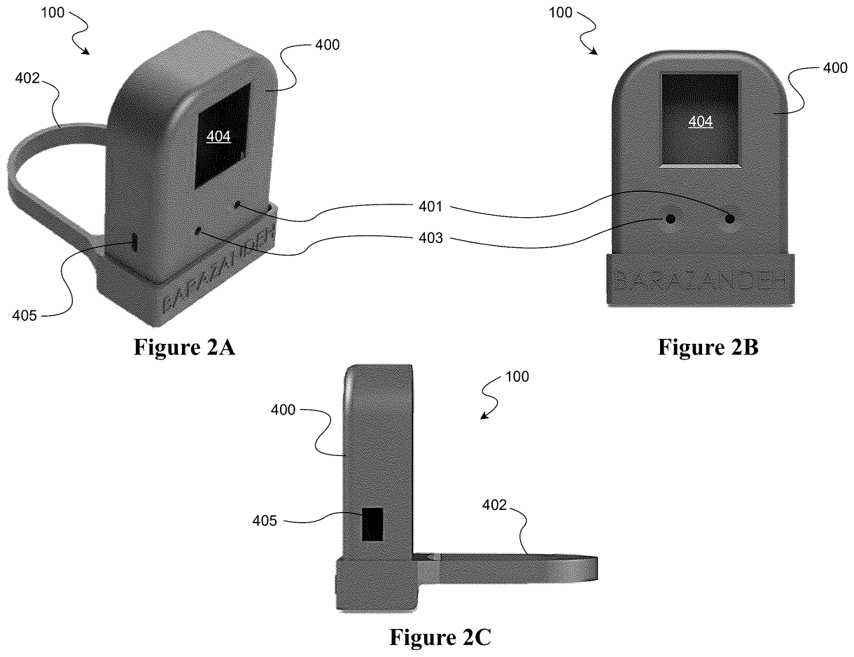

In order to improve the accuracy of measurements of the horizontal angle 13 of the slit, a high-resolution angle measuring device 100 for use in determining an angle of rotation of the device indicative of a horizontal angle 13 of rotation the slit beam is presented (see FIG. 2A). This angle measuring device 100 is couplable to and for use with slit lamps 10 in order to improve the accuracy (e.g., providing reliable measurement with a resolution of 0.1 degree) of the measuring of the horizontal angle 13 of the slit beam. The angle measuring device 100 may be integrated in a compact and easy-to-use format, and may be wirelessly connectable (e.g., via a data transceiver, such as Bluetooth®) to a user's device 444 to provide digital readings of the measurements.

One of the main aspects of the proposed angle measuring device 100 is the concept of providing a compact, affordable and easy-to-use device able to measure and determine its own angular change (i.e., its own change of angle of rotation) that is attachable to (that can be securely coupled to) an apparatus having no way of determining its angle of rotation or being limited to low-resolution angle of rotation in order to determine their common angle of rotation. In fact, being attached to one another, the provided angle measuring device 100 will share the rotation of the apparatus to which it is attached.

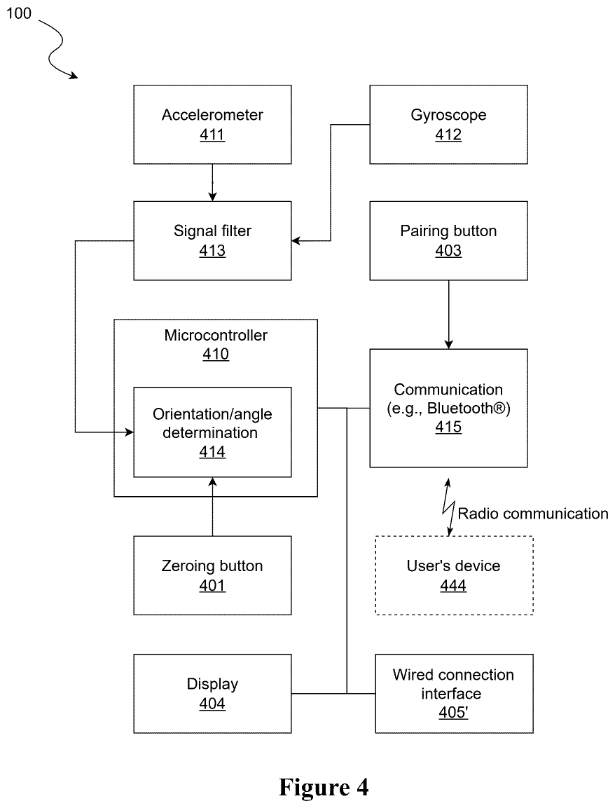

FIGS. 2A, 2B and 2C present one embodiment of the angle measuring device 100 for measuring the horizontal angle of rotation 13 of a slit beam from a slit lamp. This exemplary embodiment of the angle measuring device 100 comprises a housing 400 for hosting the various components of the angle measuring device 100 (see FIG. 4) and comprising various openings including a front opening to see the display 404.

The housing 400 of the angle measuring device 100 may have a connection port 405 used to power the angle measuring device 100 and/or for charging a battery (not shown) of the angle measuring device 100. In some embodiments, the connection port 405 may be used as a wired connection interface 405′ to connect to user's devices 444 (e.g., computing devices, smartphones, laptops, tablets, etc.) to send data (e.g., measured horizontal angle of rotation 13) thereto or exchange data therewith.

In one embodiment, the housing 400 may have a power (On/Off) switch 405 to allow a user to turn angle measuring device 100 On or Off. To extend the energy autonomy of the angle measuring device 100, the power switch 405 may be advantageous for embodiments of the angle measuring device 100 equipped with a battery.

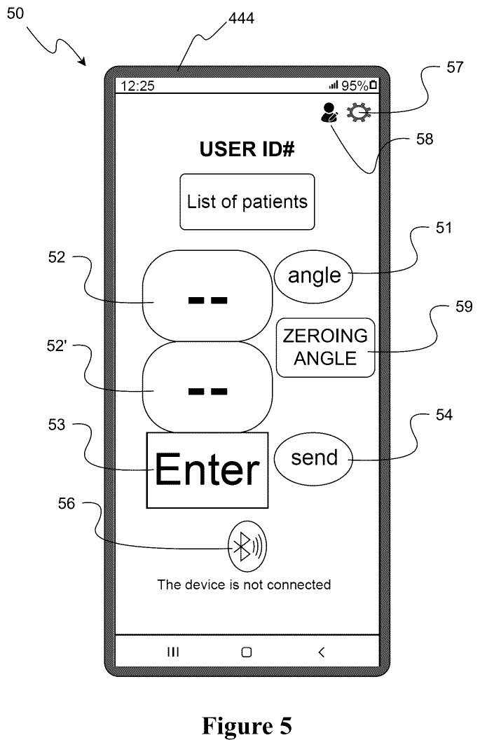

In one embodiment, the housing 400 may have an opening to allow access to a zeroing button 401 that may be used to calibrate (e.g., to indicate to the microcontroller that the present values are to be used as reference, i.e., as the zero angle value) the angle measuring device 100. In one embodiment, the housing 400 may have an opening to allow access to a pairing button 403 for wirelessly connecting a user's device 444 to the that may be used to calibrate the angle measuring device 100 (e.g., to indicate to the microcontroller that the present values are to be used as reference, i.e., as the zero angle value). Alternatively, or additionally, the zeroing and/or the pairing may be activated/triggered via a user interface (e.g., see FIG. 5) on the user's device 444.

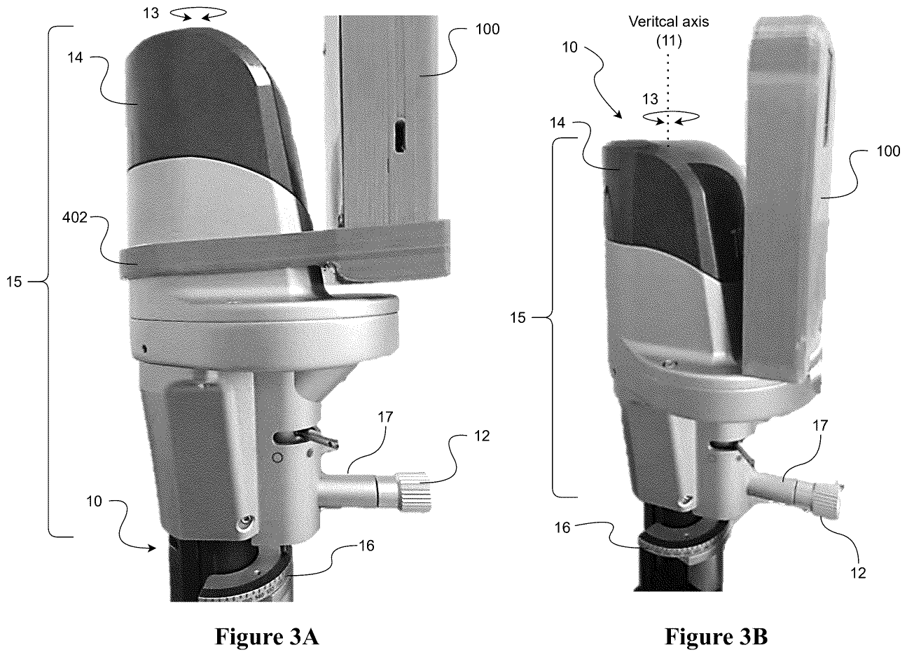

In one embodiment, the angle measuring device 100 includes a coupling member/structure 402 which may not be limited to the example of FIGS. 2A, 2B, 2C and 3A. In order to ensure that the angle measured and determined by the coupling member 402 accurately corresponds to and matches the angle of rotation of the horizontal angle of rotation 13 of the slit beam (i.e., corresponding to the rotation of the top section 15 of the slit lamp 10 around the vertical axis 11), it may be important that the coupling member 402 provides a rigid mechanical connection between the angle measuring device 100 and the top section 15 of the slit lamp 10 so as to minimize (preferably avoid) any discrepancy between the change of angle of the angle measuring device 100 and the change of angle of the top section 15 of the slit lamp 10 that would be caused by a relative shift of the angle measuring device 100 on the top section 15. It will be appreciated that these possible discrepancies may be minimized by using strong adhesives or by designing the shape of the coupling member 402 to provide an optimal fit (minimizing wiggle room) to the receiving portion of the slit lamp 10.

The coupling member 402 may be removably coupled to the slit lamp 10 or may be fixed thereto. It will be appreciated that, in some embodiments, coupling member 402 may consist of a base mounting removably or permanently couplable (for receiving and supporting) to the housing 400 of the angle measuring device 100. The base mounting 402 may be shaped to be secured to the light source housing 14 of a given model of the slit lamp 10 and configured to support the housing receivable thereby. While the coupling member 402 is primarily shown as being couplable to the light source housing 14, the person skilled in the art will appreciate that the coupling member 402 may be designed to be couplable to any other suitable portion of the top section 15.

In FIG. 3A, the angle measuring device 100 is shown installed on the top section 15 the slit lamp 10 using the coupling member 402 of FIG. 2A comprising a hoop-shape extension matching the shape of the light source housing 14 of the slit lamp 10. The coupling member 402 may be configured (e.g., shaped) to support the weight of the angle measuring device 100. Optionally, in some embodiments, the coupling member 402 may be designed to ensure that the housing 400 of the angle measuring device 100 is maintained in an upright position as shown in FIG. 3A.

It will be appreciated that the coupling member 402 may be shaped to receive, accommodate or match the shape of the light source housing. For example, the coupling member 402 may be shaped to fit at least partially (partially or fully) over or around the light source housing 14 of the slit lamp 10. In one embodiment, the coupling member 402 may be configured to be positioned and secured (fixed or removably coupled) on top of the light housing 14, which may be allowed by a shaped thereof that follows or matches as much as possible the shape of the receiving portion of the top section 15 of the slit lamp 10 (e.g., the light housing 14). The coupling member 402 may be designed to have a shape (e.g., a dome shape or a hat shape) that fully or partially covers light source housing 14 when coupled thereto. For example, the coupling member 402 may include a ring-shaped portion (as illustrated in the exemplary designs of FIGS. 2A and 3A) to follow the shape of the horizontal cross-section of the light source housing 14 at a desired position (e.g., height) thereon. The ring-shape design may be used to removably couple the angle measuring device 100 by inserting the light source housing 14 inside the ring of the coupling member 402 and by pushing the coupling member 402 down to provide a temporary tight fit therebetween. It will be appreciated that this removable coupling may be best suited when the light source housing 14 has an inverted funnel shape. While the ring-shaped coupling member may preferably have a closed ring that completely surrounds the horizontal cross section of the light source housing 14, it will be appreciated that the ring shape may have an open ring design that surrounds only a portion of the horizontal cross section using rigid (less flexible) material. It will be appreciated that the closed ring design may be best suited when using less rigid (more flexible) material like plastic for the coupling member 402.

It will be appreciated that the alternative coupling methods and/or structures may be used to attach the angle measuring device 100 to the slit lamp 10. For example, as illustrated in FIG. 3B, the angle measuring device 100 may be coupled to the slit lamp 10 by simply fixing (e.g., gluing, using Velcro, balancing on top of the lamp, etc.) thereto.

In one embodiment, the coupling member 402 may be part of the housing 400. In another embodiment, the coupling member 402 may be attachable and detachable from the housing 400. For example, the coupling member 402 fixed to the slit lamp 10 and the housing 400 may be removably attached to the coupling member 402.

Once the angle measuring device 100 is securely coupled to the top section 15 of the slit lamp 10, it can be used to track the horizontal angle of rotation 13 of the top section 15.

In one embodiment the measured horizontal angle of rotation 13 may be displayed using the display 404 of the angle measuring device 100.

In some embodiments, the angle measuring device 100 may first be calibrated by zeroing it at a desired reference angle, such that it may provide measurements of the horizontal angle of rotation of the slit beam relative to that reference angle.

It will be appreciated that zeroing step may be optional, because the high-resolution of the angle measurement may still be useful without zeroing to the reference value. In fact, the angular value of the reference angle may be separately determined/measured with high accuracy using the angle measuring device 100 before being subtracted to the angle of interest to calculate the angular difference therebetween.

Now referring to FIG. 4 that presents a block diagram of the electronic components of the proposed angle measuring device 100 for determining angles of rotation. The device 100 may comprise a microcontroller 410 (e.g., circuitry, processor/microprocessor or any combination thereof) for providing orientation/angle determination function 414. In other words, the microcontroller may execute code and/or use circuitry that acts as an orientation/angle calculator 414 for determining the angle of rotation of the device according to the received data (e.g., measurements signals from the accelerometers 411 and gyroscopes 412).

In some embodiments, the device 100 may include a signal filter 413 (e.g., a Kalman filter, such as an extended Kalman filter or an unscented Kalman filter) for filtering (e.g., preprocessing) the raw data/signals from the sensors (e.g., accelerometers 411 and gyroscopes 412) and determining robust, real-time estimations of orientation by fusing data from the inertial sensors. The filter may consist of circuitry (or analogously a processor executing program code, such as a Field-Programmable Gate Arrays (FPGAs)) having an input for receiving the signal/data from various sensors (e.g., Micro-Electro-Mechanical Systems (MEMS) sensors) and may have an output connectable or connected to the microcontroller 410 to provide angular velocity data/signal.

In one embodiment, the microcontroller 410 receives data or signals (comprising information about the angular velocity and acceleration of the device 100) from the various sensors (e.g., accelerometers 411 and gyroscopes 412) or from the signal filter 413 and uses the data/signals to determine the angle of rotation of the angle measuring device 100 based on the angular velocity and acceleration.

While the signal filter 413 is shown as an element separate from the microcontroller 410, the person skilled in the art will appreciate that the functions of the signal filter 413 (e.g., the functions of a Kalman filter) may be integrated in and provided by the microcontroller 410 itself (e.g., by the orientation/angle determination 414).

It will be appreciated that the communication features 415 provided by the microcontroller 410 may use wired communication (e.g., via the connection port 405) of wireless communication (e.g., using a data transceiver). In one embodiment the wireless communication may be provided using Bluetooth® technology (components and/or protocols).

In one embodiment, the device 100 is equipped with a dedicated a zeroing button 401 for indicating to the microcontroller a current angular value corresponds to a reference value.

In one embodiment, the device 100 may be equipped with a pairing button 403 or switch for indicating to the microcontroller 410 that the user's device is to be wirelessly connected (e.g., when pressed, the pairing button will cause the microcontroller to initiate its pairing process using wireless communication 415).

It will be appreciated the number of buttons on the device may vary as a matter of preference and may not be limited to the number or dedicated functions described in the present disclosure. In fact, in one embodiment, a single button could provide various features (e.g., On/Off, zeroing, pairing, etc.) as a function of its accessibility programming. For example, the On/Off function may be controlled when holding the button, while a single press of the button would provide the function of zeroing, and a double press would activate the pairing functions.

In some embodiments, one or more accelerometer 411 (e.g., a MEMS based accelerometer) may be provided to measure the acceleration of the angle measuring device 100 in one or more directions/axis (e.g., at least one acceleration in a single direction of rotation when the device 100 is strictly used to measure its rotation in a single dimension). In a preferred embodiment, the angle measuring device 100 comprises enough accelerometers 411 (e.g., a 9-axis accelerometer) to measure the acceleration thereof in three dimensions (i.e., at least three orthogonal directions).

In some embodiments, one or more gyroscopes 412 e.g., a MEMS gyroscope) may be provided to measure the angular velocity of the angle measuring device 100 in one or more angular orientation (e.g., at least one angular velocity in a single angular orientation when the device 100 is strictly used to measure its rotation in a single dimension). In a preferred embodiment, the angle measuring device 100 comprises enough gyroscopes 412 to measure the angular velocity thereof in three dimensions (i.e., at least three orthogonal axes of rotation).

While gyroscopes without any accelerometer may be enough to determine the desired angle of rotation 13, the preferred embodiment also comprises accelerometers to accurately determine the absolute angle of rotation, as gyroscopes measure changes in rotation (angular velocity) and drift over time. In fact, the accelerometer provides a stable reference (gravity) for orientation (pitch/roll) and helps correct for the error accumulated by the gyroscopes, especially when combined using sensor fusion algorithms like Kalman filters.

It will be appreciated that any proven method of determining the angle of rotation based on signals from one or more accelerometers 411 and one or more gyroscope 412 may be used by (coded in) the microcontroller 410 to ensure the correct and precise interpretation thereof to output a determined change of angles.

It will also be appreciated that, in most cases, the use of a single gyroscope may not be sufficient to determine the accurate angle of rotations since, depending on its position relative to the receiving slit lamp 10, the angle measuring device 100 may be orbited around a rotation axis of the slit lamp and not simply rotated around a central axis of its gyroscope. Therefore, in most cases, the combined information (acceleration and angular velocity) from the sensors (accelerometer 411 and gyroscope 412, respectively) must be considered and integrated in order to output the total angular rotation around a true rotation axis (e.g., the horizontal angle of rotation 13).

It will be appreciated that, while the accelerometer and gyroscopes are shown in FIG. 4 as separated components, these sensors may be provided and integrated in a single sensing component, such as off-the-shelf MEMS sensors that may be, but are not limited to inertial measurement units (IMUs).

The operator of the slit lamp can use the slit lamp 10 as they would normally do (e.g., adjust the horizontal angle of rotation 13 specific for the patient using the lever 17) and the angle measuring device 100 will automatically measure the horizontal angle of rotation 13. In operation, because the angle measuring device 100 is rigidly coupled to the top section 15 of the slit lamp 10, the accelerometer(s) 411 and gyroscope(s) 412 of the angle measuring device 100 will measure its acceleration and angular velocity which will be equivalent to the rotation of the top section 15.

FIG. 5 shows a schematic representation of one possible embodiment of a user interface 50, provided by a program (e.g., smartphone App), that may be displayed to the operator via their user's device 444. The user's device 444 may comprise a processor and memory storing executable program code that, when (run and display App) executed by its processor, causes the processor and the user's device 444 to provide the functions (e.g., the user interface 50, communication with the angle measuring device 100, recording determined angles, exchange data with a database or server, etc.) of the App.

The user interface 50 of FIG. 5 provides the user with the determined horizontal angle of rotation 13 for each eye of the patient (measured angle for the left eye 52 and the measure angle for the right eye 52′) relative to the reference angle (i.e., the angle at which the zeroing (e.g., using the zeroing button 401) has been completed. In some embodiments, the measured value is constantly displayed and changes, as the slit lamp 10 is being rotated, but in other embodiments the measured value is displayed only when requested (e.g., when requested to use the “angle” request button 51 of FIG. 5). In one embodiment, the selection of the measurement 53 may be completed using the user interface (e.g., using the “enter” button of FIG. 5) when the operator has completed his manipulations/adjustments of the slit lamp (e.g., selection of horizontal angle of rotation 13) for a given eye. In one embodiment, the user interface may provide the user with the option (e.g., see the “send” button of FIG. 5) of saving or sending the measured and selected values to a chosen destination (e.g., a remote database, another computing device, an internal file, a patient's personal file, etc.). In some embodiments where the user's device 444 communicates wirelessly to the angle measuring device 100, the user interface may include a directory 56 (e.g., see the Bluetooth® symbol of FIG. 5) for connecting to a known angle measuring device 100 or to set up a new connection to a new device.

In some embodiments, the App may provide via the user interface 50 a zeroing function 59 for zeroing the angle of the angle measuring device 100 using the user's device 444. This zeroing function 59 may be particularly useful and convenient to remotely zeroing the angle measuring device 100. Remotely zeroing the angle measuring device 100 may avoid physically interacting with the angle measuring device 100 or the slit lamp 10, thereby avoiding any potential change of angle during the zeroing process.

In some embodiments, the user interface may be provided by an internet web page, a dedicated application (e.g., smart phone app) or program to be executed by a computing device.

In some embodiments, the program and user interface allow the user to keep a record of patient-specific measurements (e.g., see “list of patients” in FIG. 5) to be stored in memory and accessed or modified as needed. The list of patients may be shared with all App users or may be associated with a specific user (e.g., a specific doctor) or group of users (e.g., a specific clinic).

The App may provide functions to allow the user to personalize the user profile 58 and/or the user interface 50 via the setting function/options 57.

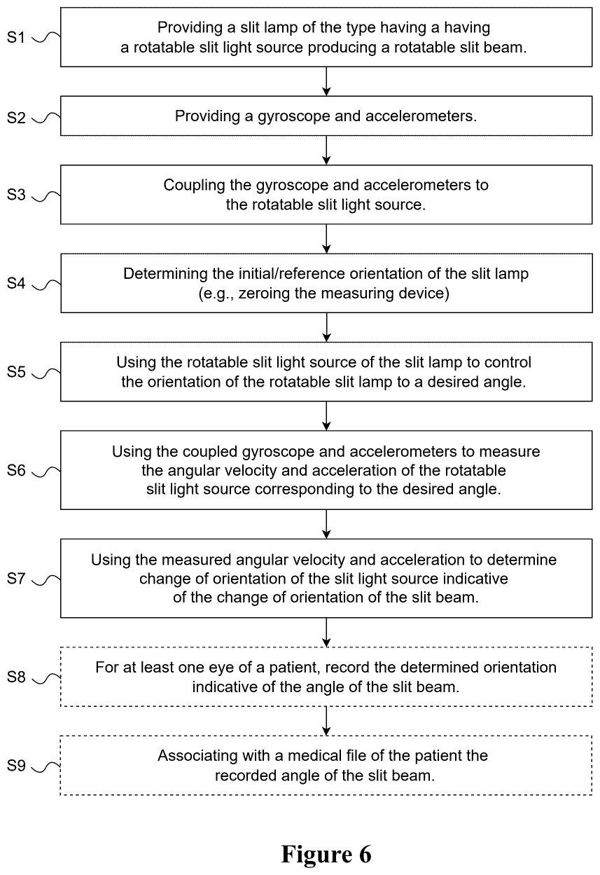

Now referring to FIG. 6 which presents a flowchart of a method to accurately measure the orientation of rotation of the slit beam using add-on gyroscopes and accelerometers. The method first calls for a first step S1 of providing a slit lamp 10 having a component being rotated in order to change the angle of a rotatable slit beam produced thereby. In one embodiment, the provided slit lamp 10 is the one of FIG. 1. In some embodiments, the slit lamp 10 comprises a slit light source 15 producing a slit beam, the slit light source 15 being mounted to be rotatable about a vertical axis 11, the slit lamp having a mirror to direct the slit beam horizontally with rotation 13 of the slit beam being about a horizontal axis.

To complete the method, the proper set of inertial sensors must be used to measure the change of orientation 13. The step S2 may be completed to provide a gyroscope 411 (e.g., MEMs gyroscope) and accelerometers 412 (e.g., MEMs accelerometers).

In step S3, the provided sensors may be attached/coupled to the slit lamp to measure changes of orientation of a component of the slit lamp 10. In one embodiment, the sensors are coupled to the slit light source 15 (upper portion) of the slit lamp 10, which has a change of orientation relative to a remainder of the slit lamp corresponding and indicative of the orientation (e.g., change of horizontal orientation 13) of the slit beam.

In order to determine the “change” of orientation 13 an initial/reference orientation must be determined and defined. While step S4 of determining the initial orientation may be manually completed by taking note of the initial/reference angle to be later subtracted to the new orientation of interest, it may be completed by zeroing the measurement values or signals from the sensors of the angle measuring device 100. It will be appreciated that, when step S4 comprises zeroing the sensors of the angle measuring device 100, step S4 is essentially implemented in and completed by a device which is distinct and does not have any human behavior equivalent since the use of the sensors and the device 100 is necessary.

The method may include a step S5 of using the rotatable slit light source of the slit lamp to control the orientation of the rotatable slit lamp to a desired angle (according to the specifics of the patient being assessed).

The method may include a step S6 of using the gyroscope and accelerometers coupled to the slit lamp to respectively measure the angular velocity and accelerations of the rotatable slit light source corresponding to the desired angle 13.

The method may include a step S7 of using the measured angular velocity and acceleration to determine change of orientation 13 of the slit light source indicative of the change of orientation of the slit beam. This may be completed using the microcontroller 410. In one embodiment, the change of orientation can be determined using a Kalman filter to process the signals and measurements.

The method may include an optional step S8 of recording the determined orientation indicative of the angle of the slit beam, which may be completed manually or using the microcontroller 410 to save in memory as data comprising information indicative of the determined change of orientation.

The method may include an optional step S9 of associating with a patient the recorded angle of the slit beam, which may be done manually or digitally (e.g., may be completed automatically) by associating data with a digital medical file of the patient.

It will be appreciated that any of the elements mentioned above may be used in any suitable combination in order to determine the desired angle. The selected elements can be provided, combined and/or used as a system 999.

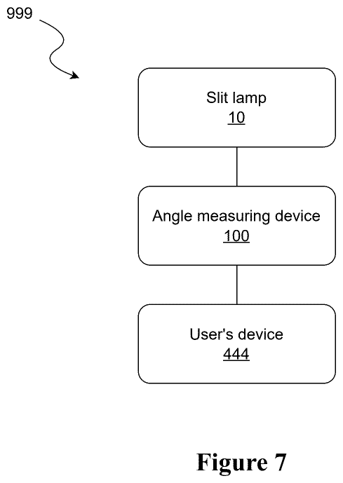

For example, FIG. 7 presents a block diagram of one embodiment of a system 999 comprising the slit lamp 10, the angle measuring device 100 and a user's device 444.

What is claimed is

1. An angle measuring device couplable to and for use with a slit lamp of the type having a slit light source producing a slit beam, the slit light source being mounted to be rotatable about a vertical axis, the slit lamp having a mirror to direct the slit beam horizontally with rotation of the slit beam being about a horizontal axis, the angle measuring device for use in determining an angle of rotation of the angle measuring device indicative of a horizontal angle of rotation of the slit beam, the angle measuring device comprising:
a housing comprising:
sensors comprising a Micro-Electro-Mechanical Systems (MEMS) gyroscope paired with MEMS accelerometers for measuring data comprising information about angular velocity and acceleration of the angle measuring device; and
a microcontroller connected the sensors for determining the angle of rotation of the angle measuring device based on the data measured by the sensors;
a coupling member shaped to be coupled to a light source outer body of the slit light source of the slit lamp and for supporting the housing; and
one of the group consisting of:
I. a display on said housing for displaying the determined angle of rotation;
II. a wireless data transceiver for providing communication to a user's device for at least transmitting data about the determined angle of rotation; and
III. a wired connection interface for outputting data about the determined angle of rotation.

2. The angle measuring device as defined in claim 1, wherein the coupling member is shaped to fit at least partially over or around the light source outer body.

3. The angle measuring device as defined in claim 2, wherein the coupling member comprises a ring-shaped portion for removably coupling to the light source outer body and housing coupling portion for coupling to and supporting the housing.

4. The angle measuring device as defined in claim 1, wherein the angle measuring device comprises the data transceiver, wherein the data transceiver uses Bluetooth® technology.

5. The angle measuring device as defined in claim 4, wherein the angle measuring device comprises the data transceiver, and wherein the angle measuring device further comprises a pairing button for indicating to the microcontroller that the user's device is to be wirelessly connected.

6. The angle measuring device as defined in claim 1, further comprising a zeroing button for indicating to the microcontroller a current angular value corresponds to a reference value.

7. The angle measuring device as defined in claim 1, wherein a Kalman filter is associated with the microcontroller for determining the angle of rotation of the angle measuring device.

8. The angle measuring device as defined in claim 1, wherein the sensors comprise a nine-axis accelerometer.

9. The angle measuring device as defined in claim 1, wherein the coupling member is removably couplable to the outer body or removably couplable to the light source outer body of the slit light source of the slit lamp.

10. The angle measuring device as defined in claim 1, wherein the coupling member comprises a base mounting having a shape to receive, accommodate or match the shape of the light source outer body.

11. The angle measuring device as defined in claim 1, wherein the sensors comprise an inertial measurement unit (IMU).

12. The angle measuring device as defined in claim 1, further comprising a signal filter connected to the sensors to receive the measured data and connected to the microcontroller to provide at least a yaw angular rotation of the angle measuring device.

13. A system for determining a horizontal angle of rotation of a slit beam of a slit lamp, the system comprising:
a slit lamp of the type having a slit light source producing the slit beam, the slit light source being mounted to be rotatable about a vertical axis, the slit lamp having a mirror to direct the slit beam horizontally with rotation of the slit beam being about a horizontal axis;
an angle measuring device couplable to the slit lamp, the angle measuring device comprising:
a housing comprising:
sensors comprising a Micro-Electro-Mechanical Systems (MEMS) gyroscope paired with MEMS accelerometers for measuring data comprising information about angular velocity and acceleration of the angle measuring device; and
a microcontroller connected the sensors for determining the angle of rotation of the angle measuring device based on the data measured by the sensors;
a coupling member shaped to be coupled to a light source outer body of the slit light source of the slit lamp and for supporting the housing; and
one of the group consisting of:
I. a display for displaying the determined angle of rotation of the angle measuring device,
II. a wireless data transceiver for providing communication to a user's device for at least transmitting the determined angle of rotation of the angle measuring device, and
III. a wired connection interface for outputting the determined angle of rotation of the angle measuring device; and
a user's device for receiving, from the angle measuring device, data about the angle of rotation determined by the microcontroller of the angle measuring device and comprising a processor and memory comprising executable program code that, when executed by the processor, causes the processor to provide a user interface displaying the angle of rotation determined by the microcontroller of the angle measuring device.

14. The system of claim 13, wherein the program code, when executed by the processor, further causes the user's device to activate an angle zeroing function of the angle measuring device so a current angular value determined by the angle measuring device corresponds to a reference value.

15. A method of determining an angle of rotation of a slit light source of a slit lamp indicative of a horizontal angle of rotation a slit beam of the slit lamp, the method comprising:
providing the slit lamp, wherein the slit lamp is of the type having the slit light source producing the slit beam, the slit light source being mounted to be rotatable about a vertical axis, the slit lamp having a mirror to direct the slit beam horizontally with rotation of the slit beam being about a horizontal axis;
providing a Micro-Electro-Mechanical Systems (MEMS) gyroscope and MEMS accelerometers;
coupling the MEMS gyroscope and MEMS accelerometers to the slit light source;
determining an initial orientation of slit light source corresponding to a reference orientation of the slit lamp;
rotating the slit light source around the vertical axis to change an orientation of the slit beam;
using the coupled MEMS gyroscope and MEMS accelerometers to measure the angular velocity and acceleration during said rotating of the slit light source around the vertical axis; and
using the measured angular velocity and the measured acceleration to determine the change of orientation of the slit light source of the slit lamp indicative of the horizontal angle of rotation the slit beam of the slit lamp.

16. The method as defined in claim 15, wherein the step of determining the initial orientation of slit light source comprises positioning the slit light source to the initial orientation and zeroing the MEMS gyroscope and the MEMS accelerometers.

17. The method as defined in claim 15, further comprising recording the determined change of orientation.

18. The method as defined in claim 17, wherein the recording the determined change of orientation comprises saving in memory data comprising information indicative of the determined change of orientation.

19. The method as defined in claim 17, further comprising associating the recorded change of orientation with a patient.

20. The method as defined in claim 17, further comprising associating the data with a digital medical file of the patient.
